(19) United States
(12) Patent Application Publication
(10) Pub. No.: US 2009/0300783 A1
(43) Pub. Date: Dec. 3, 2009

(54) METHODS FOR MAKING AND USING WHEAT PLANTS WITH INCREASED GRAIN PROTEIN CONTENT

(76) Inventors: William J. Howie, Cary, NC (US); Ronald E. Kehler, Oakville (CA); Dale R. Carlson, Carrboro, NC (US); Mark L. Dahmer, Centennial, CO (US); Bijay K. Singh, Cary, NC (US)

Correspondence Address: ALSTON & BIRD LLP;BASF CORPORATION, BANK OF AMERICA PLAZA, 101 SOUTH TRYON STREET, SUITE 4000, CHARLOTTE, NC, 28280-4000, US

(21) Appl. No.: 12/302,101

(22) PCT Filed: May 31, 2007
(86) PCT No.: PCT/US07/70070

§ 371 Date: Apr. 24, 2009

Related U.S. Application Data
(60) Provisional application No. 60/809,722, filed on May 31, 2006.

(51) Int. Cl. C12N 15/82; A01H 1/02

(52) U.S. Cl. 800/260; 800/278

(57) ABSTRACT

The present invention provides novel methods for making wheat plants with increased grain protein content. The methods involve introducing a gene encoding herbicide-resistant, wheat acetohydroxyacid synthase large subunit (AHASL) protein. The invention further provides wheat plants that produce high protein grain and human and animal food products derived thereof.

### FIELD OF THE INVENTION

This invention relates to the field of agricultural, particularly to novel methods for making and using wheat plants with increased grain protein content.

### BACKGROUND OF THE INVENTION

Grain protein content of wheat is important for both the improvement of the nutritional value and also is a major contributory factor for making bread (Dick & Youngs (1988) “Evaluation of durum wheat, semolina, and pasta in the United States,” In: Durum wheat: Chemistry and technology, AACC, St. Paul, Minn., pp. 237-248; Finney et al (1987) “Quality of hard, soft, and durum wheats”. In E. G. Heyne (ed.) Wheat and wheat improvement, Agron. Monogr. 13, 2nd ed. ASA, CSSA, and SSSA, Madison, Wis., pp. 677-748; Khan et al. (2000) Crop Sci. 40:518-524). It is also an important trait for growers due to the premium price for wheat with high grain protein (Olmos et al. (2003) Theor. Appl. Genet. 107:1243-1251). Breeding for high grain protein content has received a lot of effort but progress has been slow due to complexity of the genetics controlling the trait and interaction with environment. Studies have identified several QTLs for grain protein content (Turner, et al. (2004) J. Cereal Sci. 40:51-60; Joppa et al. (1997) Crop Sci. 37: 1586-158; Perretant et al. (2000) Theor. Appl. Genet. 100:1167-1175; Prasad et al. (1999) Theor. Appl. Genet. 99:341-345; Groos et al. (2003) Theor. Appl. Genet. 106:1032-1040; Groos et al. (2004) J. Cereal Sci. 40:93-100; Shewry et al. (1997) J. Sci. Food Agric. 73:397-406). An improvement of grain protein content by 1 to 2% was considered as significant increase within a given class or type of wheat (Tokatilidis et al. (2004) Field Crops Res. 86:33-42; Olmos et al. (2003) Theor. Appl. Genet. 107:1243-1251; Mesfin et al. (2000) Euphytica 116:237-242). Grain protein-content is influenced by environmental conditions such as soil fertility, temperature, nitrogen nutrition, rainfall or temperature (Bhullar & Jenner (1985) Aust. J. Plant Physiol. 12: 363-375; Wardlaw & Wrigely (1994) Aust. J. Plant Physiol. 21:695-703; Daniel & Triboi (2000) J. Cereal Sci. 32: 45-56; Metho et al. (1999) J. Sci. Food Agric. 79:1823-1831). Research has also shown there is a negative effect of high protein on yield (Cox et al. (1985) Crop Sci. 25:430-435; Day et al. (1985) J. Plant Nutrition 8:555-566); however others suggest that it should be possible to breed wheat with both traits (Day et al. (1985) J. Plant Nutrition 8:555-566; Johnson et al. (1978) “Breeding progress for protein and lysine in wheat,” In: Proceedings of the Fifth International Wheat Genetics Symposium, New Delhi, India, pp. 825-835). Certainly, having a single gene trait or closely linked traits affecting grain protein would provide significant advantages improving both the bread making and nutritional value of bread wheat, particularly if the trait or closely linked traits allow for quick and cost-effective selection.

### SUMMARY OF THE INVENTION

The present invention provides methods for making wheat plants that produce grain with increased grain protein content. The invention is based on the surprising discovery that wheat plants which comprise in their genomes at least one copy of an AHASL1A gene that encodes an AHASL1A protein comprising a serine-to-asparagine substitution at amino acid position 579 in the Triticum aestivum AHASL1A protein. This amino acid substitution is also referred to herein as the S653N substitution because the corresponding serine-to-asparagine substitution is at amino acid position 653 in the Arabidopsis thaliana AHASL1 protein. The methods of the invention involve introducing at least one copy of a wheat AHASL1A gene that encodes an AHASL1A protein comprising the S653N substitution into a plant. Such a gene can be introduced by methods such as, for example, cross pollination, mutagenesis, and transformation. The methods of the invention can further involve growing the wheat plant or a descendent plant thereof comprising the AHASL1A S653N gene to produce grain and determining the protein content of grain produced by the wheat plant or the descendent plant. The methods can additionally involve selecting for plants that comprise the wheat AHASLA1 S653N gene by, for example, applying an effective amount of an AHAS-inhibiting herbicide to the plant and/or to the soil or other substrate in which the plant is growing or will be grown.

The present invention further provides wheat plants, plant organs, plant tissues, and plants cells, and high protein grain as well as human and animal food products derived from the high protein grain produced by the wheat plants of the invention. Methods of using the high protein grain of the invention to produce food products for humans and animals are also provided.

### BRIEF DESCRIPTION THE DRAWING

FIG. 1 is a graphical representation of the results of an in vitro investigation to determine the feedback inhibition of AHAS activity by valine and leucine using enzyme extracts prepared from wheat plants of the BW255-2 and control BW255 lines. The BW255-2 line is homozygous for the AHASL1A S653N allele. The BW255 is homozygous wild-type at AHASL1A gene and is the parental line that was mutagenized to produce the BW255-2 line.

### DETAILED DESCRIPTION OF THE INVENTION

The present invention provides methods for making and using wheat plants that comprise grain with increased grain protein content. The invention involves introducing into a wheat plant at least one copy of a wheat AHASL1A gene that encodes an AHASL1A protein comprising the S653N substitution into a plant. Such a gene can be introduced by methods such as, for example, cross pollination, mutagenesis, and transformation. The methods of the invention can further involve growing the wheat plant or a descendent plant thereof comprising the AHASL1A S653N gene to produce grain and determining the protein content of grain produced by the wheat plant or the descendent plant. The wheat plants produced by the methods of the present invention and the descendent plants thereof comprise an increased grain protein content when compared to wheat plants lacking the wheat AHASL1A S653N gene.

The methods of the present invention find use in the development of new wheat cultivars with increased grain protein content. When compared to existing methods, the methods of invention considerably decrease the breeding effort required to develop high protein wheat because the methods of the invention provide for a robust selection advantage due to the high protein wheat trait being linked to an easily selectable herbicide tolerance trait. Furthermore, selectable molecular markers are known in the art for the wheat AHASL1A S653N gene and thus, can aid in marker-assisted breeding approaches for wheat with increased grain protein content. See, U.S. Pat. App. Pub. No. 2005/0208506, herein incorporated by reference. In addition to the advantages provided by the ease of selection for the high protein trait, the increase in grain protein content is not correlated with a loss in grain yield. Thus, the methods of the invention provide wheat plants that produce grain with increased protein content and these plants can be used to increase the amount of grain protein produced per acre as compared to similar wild-type plants. Finally the high protein trait of the present invention can be combined with existing bread wheat germplasm that is already high in grain protein content to develop wheat lines with even higher grain protein content.

The present invention provides high protein wheat plants and the high protein grain produced by these plants. Such high protein grain finds use in a variety of food and feed products for human and animal consumption. In particular, the grain produced by wheat plants of the invention finds use in the production of high protein wheat flour, particularly for use in bread making. Thus, the invention provides methods for making high protein flour comprising milling grain produced by the high protein wheat plants of the present invention.

The high protein wheat plants of the invention also comprise increased resistance to herbicides when compared to a wild-type wheat plant. In particular, the high protein wheat plants of the invention have increased resistance to at least one herbicide that interferes with the activity of the AHAS enzyme when compared to a wild-type wheat plant. The high protein wheat plants of the invention comprise at least one copy of a wheat AHASL1 S653N gene or polynucleotide. Such a wheat AHASL1A protein comprises an asparagine at amino acid position 579 or equivalent position. In the wild-type AHASL1A protein, a serine is found at position 579. Because the corresponding position in the well-known AHASL1 protein of Arabidopsis thaliana is amino acid 653, the AHASL1A gene encoding the AHASL1A protein comprising the serine₅₇₉-to-asparagine substitution is referred to as the AHASL1A S653N gene to conform to the established nomenclature for plant AHASL sequences.

The present invention provides methods for making wheat plants that comprise grain with increased grain protein content. In one embodiment, the methods involves introducing into a wheat plant at least one copy of a wheat AHASL1A gene that encodes an AHASL1A protein comprising the S653N substitution into a plant by mutagenesis, particularly by mutagenizing an endogenous wheat AHASL1A gene to produce a wheat AHASL1A S653 gene. Any mutagenesis method known in the art may be used to produce the high protein wheat plants of the present invention. Such mutagenesis methods can involve, for example, the use of any one or more of the following mutagens: radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as ethyl methanesulfonate (EMS), base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethyleneamines, sulfates, sulfonates, sulfones, lactones), azide, hydroxylamine, nitrous acid, or acridines. Wheat plants comprising a wheat AHASL1A S653N gene can also be produced by using tissue culture methods to select for plant cells comprising herbicide-resistance mutations, selecting for plants comprising a AHASL1A S653N gene, and regenerating plants therefrom. See, for example, U.S. Pat. Nos. 5,773,702 and 5,859,348, both of which are herein incorporated in their entirety by reference. Further details of mutation breeding can be found in “Principals of Cultivar Development” Fehr, 1993 Macmillan Publishing Company the disclosure of which is incorporated herein by reference.

In one embodiment, the present invention provides high protein wheat plants that comprise one, two, three, four, or more copies of the wheat AHASL1A S653N gene or polynucleotide. For example, a high protein wheat can comprise one or two copies of the AHASL1A S653N gene at the native wheat AHASL1A locus and can additionally or alternatively comprise one, two, three, or more copies of AHASL1A S653N polynucleotide that is operably linked to the native wheat AHASL1A promoter or to another promoter capable of driving expression in a plant, particularly during grain fill, such as, for example, a seed-preferred or an embryo-preferred promoter.

The present invention provides methods for making wheat plants that comprise grain with increased grain protein content. In an embodiment of the invention, the methods comprise transforming a plant cell with a polynucleotide construct comprising a nucleotide sequence operably linked to a promoter that drives expression in a plant cell and regenerating a transformed plant from the transformed plant cell. The nucleotide sequence encodes a wheat AHASL1A protein comprising an asparagine at amino acid position 579 or equivalent position. Nucleotide sequences encoding wheat AHASL proteins and wheat plants comprising the wheat AHASL1A S653N gene have been previously disclosed. See, WO 2004/106529 and U.S. Patent Application Publication Nos. 2004/0237134 2004/0244080, 2005/0044597, 2006/0010514, and 2006/0095992; all of which are herein incorporated by reference. In other embodiments, the methods involve conventional plant breeding involving cross pollination of a wheat plant comprising at least one copy of the wheat AHASL1A S653N gene with another wheat plant and may further involve selecting for progeny plants (F1 or F2) that comprise the herbicide-resistance characteristics of the parent plant that comprises a AHASL1A S653N gene. The methods can optionally involve self-pollination of the F1 plants and selection for subsequent progeny plants (F2) so as to produce wheat lines that are homozygous for AHASL1A S653N. If desired, the methods can further involve the self-pollination of one or more subsequent generations (i.e., F2, F3, F4, etc.) and selection for subsequent progeny plants (i.e., F3, F4, F5, etc.) that are homozygous for AHASL1A S653N. Unless expressly stated or otherwise apparent from the context of use, the term “progeny” as used herein is not limited to the immediate offspring of a plant but includes descendents from subsequent generations.

The methods of the present invention involve the use of wheat plants comprising at least one wheat AHASL1A S653N gene. Such wheat plants include, but are not limited to: a wheat plant deposited with the American Type Culture Collection, Manassas, Va. 20110-2209 USA on Jan. 15, 2002 under Patent Deposit Designation Number PTA-3955, Patent Deposit Designation Number PTA-4113, deposited with American Type Culture Collection, Manassas, Va. 20110-2209 US on Mar. 19, 2002; and Patent Deposit Designation Number PTA-4257, deposited with American Type Culture Collection, Manassas, Va. 20110-2209 US on May 28, 2002; a mutant, recombinant, or genetically engineered derivative of the wheat plant with ATCC Patent Deposit Designation Number PTA-3955, PTA-4113, and/or PTA-4257; any descendents of the plant with ATCC Patent Deposit Designation Number PTA-3955, PTA-4113, and/or PTA-4257; and a wheat plant that is the descendent of any one or more of these plants. Preferably, such mutant, recombinant, or genetically engineered derivatives of any of the wheat plants having ATCC Patent Deposit Designation Number PTA-3955, PTA-4113, and PTA-4257, and descendent thereof comprise the herbicide resistance characteristics of the wheat plant having ATCC Patent Deposit Designation Number PTA-3955, PTA-4113, or PTA-4257. The wheat plants having ATCC Patent Deposit Designation Number PTA-3955, PTA-4113, and PTA-4257, and derivatives and descendent thereof are described in U.S. Patent Application Publication Nos. 2004/0237134, 2004/0244080, and 2006/0095992; all of which are herein incorporated by reference.

A deposit of at least 2500 seeds for each of the wheat lines having ATCC Patent Deposit Designation Numbers PTA-3955, PTA-4113, and PTA-4257 was made with the Patent Depository of the American Type Culture Collection, Manassas, Va. 20110 USA on Jan. 3, 2002, Mar. 4, 2002, and Jan. 3, 2002, respectively. Each of these deposits was made for a term of at least 30 years and at least 5 years after the most recent request for the furnishing of a sample of the deposit is received by the ATCC. These deposits will be maintained under the terms of the Budapest Treaty on the International Recognition of the Deposit of Microorganisms for the Purposes of Patent Procedure. Additionally, these deposits satisfy all requirements of 37 C.F.R. §§ 1.801-1.809, including providing an indication of the viability of the sample.

As used herein, unless indicated otherwise or apparent from the context, the term “plant” includes, but is not limited to, plant cells, plant protoplasts, plant cell tissue cultures from which plants can be regenerated, plant calli, plant clumps, plant cells that are intact in plants, or parts of plants such as, for example, embryos, pollen, ovules, cotyledons, leaves, stems, flowers, branches, petioles, fruit, roots, root tips, anthers, and the like. Furthermore, it is recognized that a seed is a plant.

A “high protein wheat plant” is intended to mean a wheat plant produced by the methods disclosed herein that produces or is capable of producing grain with grain protein content levels that are increased over the level of a similar wheat plant that does not comprise in its genome at least one copy of a wheat AHASL1A S653N gene of the present invention. In a preferred embodiment of the invention, the high protein wheat plants are Triticum aestivum wheat plants.

Grain produced by the high protein wheat plants of the invention is referred to herein as “high protein grain.”

The “high protein trait” of the present invention is high grain protein content and is due to the presence of the wheat AHASL1A S653N gene or polynucleotide of the present invention in the genome of a wheat plant. Such AHASL1A S653N genes include the AHASL1A S653N genes from any wheat species that possesses the A genome, including, but not limited to, Triticum aestivum L., T. monococcum L., T. turgidum L. (including, but not limited to subsp. carthlicum, durum, dicoccoides, dicoccum, polonicum, and turanicum), and T. spelta L.

The present invention provides high protein wheat plants that produce grain with increased grain protein content. Typically, grain protein content is determined as a percentage of the weight of mature, dry grain. Generally, the protein content of grain produced by the wheat plants of the present invention is at least about 4, 5, 6, or 7% higher than similar control wheat plants that do not comprise at least one copy of a wheat AHASL1A S653N gene. Preferably, the protein content of grain produced by the wheat plants of the present invention is at least about 8, 9, 10, or 11% higher than similar control wheat plants. More preferably, the protein content of grain produced by the wheat plants of the present invention is at least about 12, 13, 14, or 15% higher than similar control wheat plants. Even more preferably, the protein content of grain produced by the wheat plants of the present invention is at least about 15, 16, 17, or 18% higher than similar control wheat plants. Still even more preferably, the protein content of grain produced by the wheat plants of the present invention is at least about 19, 20, 21, or 22% higher than similar control wheat plants. Most preferably, the protein content of grain produced by the wheat plants of the present invention is at least about 23% higher than similar control wheat plants.

The present invention does not depend on any particular methods for determining grain protein content or other grain components such as moisture content and the levels of individual amino acids. Any methods know in the art can be used to determine grain protein content, moisture and individual amino acids. See, for example, Official Methods of Analysis of AOAC International (2005), 18th Ed., AOAC International, Gaithersburg, Md., USA, Official Methods 990.03 (crude protein), 930.15 (moisture), and 982.30 (amino acids/protein efficiency ratio); herein incorporated by reference.

As used herein, a “derivative” of a plant or a “derivative wheat plant” is a wheat plant that is a descendent or clone of a high protein wheat plant of the present invention and comprises at least one copy of a wheat AHASL1A S653N gene that was inherited from the high protein wheat plant and is also a high protein wheat plant as defined herein, unless indicated otherwise or apparent from the context. Such derivatives or derivative wheat plants include descendents of a high protein wheat plant that result for sexual and/or asexual reproduction and thus, include both non-transgenic and transgenic wheat plants.

The present invention is directed to high protein wheat plants that are herbicide-tolerant or herbicide-resistant wheat plants. By an “herbicide-tolerant” or “herbicide-resistant” plant, it is intended that a plant that is tolerant or resistant to at least one herbicide at a level that would normally kill, or inhibit the growth of, a normal or wild-type plant. The high protein wheat plants of the invention comprise a herbicide-tolerant or herbicide-resistant AHASL protein, particularly a AHASL1A S653N. By “herbicide-tolerant AHASL protein” or “herbicide-resistant AHASL protein”, it is intended that such an AHASL protein displays higher AHAS activity, relative to the AHAS activity of a wild-type AHASL protein, when in the presence of at least one herbicide that is known to interfere with AHAS activity and at a concentration or level of the herbicide that is to known to inhibit the AHAS activity of the wild-type AHASL protein. Furthermore, the AHAS activity of such a herbicide-tolerant or herbicide-resistant AHASL protein may be referred to herein as “herbicide-tolerant” or “herbicide-resistant” AHAS activity.

For the present invention, the terms “herbicide-tolerant” and “herbicide-resistant” are used interchangeable and are intended to have an equivalent meaning and an equivalent scope. Similarly, the terms “herbicide-tolerance” and “herbicide-resistance” are used interchangeable and are intended to have an equivalent meaning and an equivalent scope. Likewise, the terms “imidazolinone-resistant” and “imidazolinone-resistance” are used interchangeable and are intended to be of an equivalent meaning and an equivalent scope as the terms “imidazolinone-tolerant” and “imidazolinone-tolerance”, respectively.

The invention encompasses the use or herbicide-resistant wheat AHASL polynucleotides and herbicide-resistant wheat AHASL proteins, particularly wheat AHASL1A S653N genes or polynucleotides and wheat AHASL1A S653N proteins. By “herbicide-resistant AHASL polynucleotide” is intended a polynucleotide that encodes a protein comprising herbicide-resistant AHAS activity. By “herbicide-resistant AHASL protein” is intended a protein or polypeptide that comprises herbicide-resistant AHAS activity.

Further, it is recognized that a herbicide-tolerant or herbicide-resistant AHASL protein can be introduced into a plant by transforming a plant or ancestor thereof with a nucleotide sequence encoding a herbicide-tolerant or herbicide-resistant AHASL protein. Such herbicide-tolerant or herbicide-resistant AHASL proteins are encoded by the herbicide-tolerant or herbicide-resistant AHASL polynucleotides. Alternatively, a herbicide-tolerant or herbicide-resistant AHASL protein may occur in a plant as a result of a naturally occurring or induced mutation in an endogenous AHASL gene in the genome of a plant or progenitor thereof.

The present invention provides high protein wheat plants and plant tissues, plant cells and grain thereof that comprise tolerance to at least one herbicide, particularly a herbicide that interferes with the activity of the AHAS enzyme, more particularly an imidazolinone or sulfonylurea herbicide. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, microspore, or host cell, but that said amount does not kill or inhibit as severely the growth of the herbicide-resistant plants, plant tissues, plant cells, microspores, and host cells of the present invention. Typically, the effective amount of a herbicide is an amount that is routinely used in agricultural production systems to kill weeds of interest. Such an amount is known to those of ordinary skill in the art, or can be easily determined using methods known in the art. Furthermore, it is recognized that the effective amount of a herbicide in an agricultural production system might be substantially different than an effective amount of a herbicide for a plant culture system such as, for example, the microspore culture system described below in Example 1.

The herbicides of the present invention are those that interfere with the activity of the AHAS enzyme such that AHAS activity is reduced in the presence of the herbicide. Such herbicides may also referred to herein as “AHAS-inhibiting herbicides” or simply “AHAS inhibitors.” As used herein, an “AHAS-inhibiting herbicide” or an “AHAS inhibitor” is not meant to be limited to single herbicide that interferes with the activity of the AHAS enzyme. Thus, unless otherwise stated or evident from the context, an “AHAS-inhibiting herbicide” or an “AHAS inhibitor” can be a one herbicide or a mixture of two, three, four, or more herbicides, each of which interferes with the activity of the AHAS enzyme.

By “similar, wild-type wheat plant” is intended a wheat plant that lacks the high protein grain and herbicide-resistance traits that are disclosed herein. The use of the term “wild-type” is not, therefore, intended to imply that a plant, plant tissue, plant cell, or other host cell lacks recombinant DNA in its genome, and/or does not possess herbicide resistant characteristics that are different from those disclosed herein.

The plants of the present invention include both non-transgenic plants and transgenic plants. By “non-transgenic plant” is intended mean a plant lacking recombinant DNA in its genome. By “transgenic plant” is intended to mean a plant comprising recombinant DNA in its genome. Such a transgenic plant can be produced by introducing recombinant DNA into the genome of the plant. When such recombinant DNA is incorporated into the genome of the transgenic plant, progeny of the plant can also comprise the recombinant DNA. A progeny plant that comprises at least a portion of the recombinant DNA of at least one progenitor transgenic plant is also a transgenic plant.

The present invention involves wheat plants comprising AHASL1A proteins with an amino acid substitution at a amino acid position 579, which is within a known conserved region of the wheat AHASL1A protein. See, Table 4 below. Those of ordinary skill will recognize that such amino acid positions can vary depending on whether amino acids are added to or removed from, for example, the N-terminal end of an amino acid sequence. Thus, the invention encompasses wheat AHASL1A protein with amino substitutions at the recited position or equivalent position (e.g., “amino acid position 579 or equivalent position”). By “equivalent position” is intended to mean a position that is within the same conserved region as the exemplified amino acid position. See, Table 4 below. Because the position that is equivalent to amino aid 579 of the wheat AHASL1A protein is amino acid 653 of the Arabidopsis thaliana AHASL1 protein, the wheat AHASL1A protein with the serine to asparagine substitution at amino acid position 579 is also referred to herein as the wheat AHASL1A S653N protein to conform to the well accepted nomenclature in the field of the present invention that is based on the amino acid sequence of the Arabidopsis thaliana AHASL1 protein. Similarly, the gene or polynucleotide encoding the wheat AHASL1A S653N protein is referred to herein as the wheat AHASL1A S653N gene or the wheat AHASL1A S653N polynucleotide.

The present invention is drawn to high protein wheat plants comprising enhanced tolerance or resistance to at least one herbicide that interferes with the activity of the AHAS enzyme. Such AHAS-inhibiting herbicides include imidazolinone herbicides, sulfonylurea herbicides, triazolopyrimidine herbicides, pyrimidinyloxybenzoate herbicide, sulfonylamino-carbonyltriazolinone herbicides, or mixture thereof. Preferably, the AHAS-inhibiting herbicide is an imidazolinone herbicide. For the present invention, the imidazolinone herbicides include, but are not limited to, PURSUIT® (imazethapyr), CADRE® (imazapic), RAPTOR® (imazamox), SCEPTER® (imazaquin), ASSERT® (imazethabenz), ARSENAL® (imazapyr), a derivative of any of the aforementioned herbicides, and a mixture of two or more of the aforementioned herbicides, for example, imazapyr/imazamox (ODYSSEY®). More specifically, the imidazolinone herbicide can be selected from, but is not limited to, 2-(4-isopropyl-4-methyl-5-oxo-2-imidazolin-2-yl)-nicotinic acid, [2-(4-isopropyl)-4-][methyl-5-oxo-2-imidazolin-2-yl)-3-quinolinecarboxylic]acid, [5-ethyl-2-(4-isopropyl-]4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, [2-(4-isopropyl-4-methyl-5-oxo-2-]imidazolin-2-yl)-5-methylnicotinic acid, and a mixture of methyl[6-(4-isopropyl-4-]methyl-5-oxo-2-imidazolin-2-yl)-m-toluate and methyl[2-(4-isopropyl-4-methyl-5-]oxo-2-imidazolin-2-yl)-p-toluate. The use of 5-ethyl-2-(4-isopropyl-4-methyl-5-oxo-2-imidazolin-2-yl)-nicotinic acid and [2-(4-isopropyl-4-methyl-5-oxo-2-imidazolin-2-]yl)-5-(methoxymethyl)-nicotinic acid is preferred. The use of [2-(4-isopropyl-4-]methyl-5-oxo-2-imidazolin-2-yl)-5-(methoxymethyl)-nicotinic acid is particularly preferred.

Sulfonylurea herbicides include, but are not limited to, chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfiuon, fluzasulfuron, imazosulfuron, pyrazosulfuron ethyl, halosulfuron, azimsulfuron, cyclosulfuron, ethoxysulfuron, flazasulfuron, flupyrsulfuron methyl, foramsulfuron, iodosulfuron, oxasulfuron, mesosulfuron, prosulfuron, sulfosulfuron, trifloxysulfuron, tritosulfuron, a derivative of any of the aforementioned herbicides, and a mixture of two or more of the aforementioned herbicides. The triazolopyrimidine herbicides of the invention include, but are not limited to, cloransulam, diclosulam, florasulam, flumetsulam, metosulam, and penoxsulam. The pyrimidinyloxybenzoate herbicides of the invention include, but are not limited to, bispyribac, pyrithiobac, pyriminobac, pyribenzoxim and pyriftalid. The sulfonylamino-carbonyltriazolinone herbicides include, but are not limited to, flucarbazone and propoxycarbazone.

It is recognized that pyrimidinyloxybenzoate herbicides are closely related to the pyrimidinylthiobenzoate herbicides and are generalized under the heading of the latter name by the Weed Science Society of America. Accordingly, the herbicides of the present invention further include pyrimidinylthiobenzoate herbicides, including, but not limited to, the pyrimidinyloxybenzoate herbicides described above.

The present invention provides methods for producing a high protein wheat plant involving the introduction into the genome of a wheat plant at least one copy of a wheat AHASL1A S653N gene so as to produce a high protein wheat plant. In one embodiment of the invention, at least one copy of a wheat AHASL1A S653N gene is introduced into a wheat plant by transforming the wheat plant with a polynucleotide construct comprising a promoter operably linked to a wheat AHASL1A S653N polynucleotide sequence of the invention. The methods involve introducing the polynucleotide construct of the invention into at least one plant cell and regenerating a transformed plant therefrom. The methods further involve the use of a promoter that is capable of driving gene expression in a plant cell. Preferably, such a promoter is a promoter that drives expression in the developing wheat grain, particularly during the time when protein accumulation is known to occur. Such promoters include, for example, constitutive promoters and seed-preferred promoters. A wheat plant produced by this method comprises increased AHAS activity, particularly herbicide-tolerant AHAS activity, and increase grain protein content, when compared to a similar untransformed wheat plant.

The use of the term “polynucleotide constructs” herein is not intended to limit the present invention to polynucleotide constructs comprising DNA. Those of ordinary skill in the art will recognize that polynucleotide constructs, particularly polynucleotides and oligonucleotides, comprised of ribonucleotides and combinations of ribonucleotides and deoxyribonucleotides may also be employed in the methods disclosed herein. Thus, the polynucleotide constructs of the present invention encompass all polynucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The polynucleotide constructs of the invention also encompass all forms of polynucleotide constructs including, but not limited to, single-stranded forms, double-stranded forms, hairpins, stem-and-loop structures, and the like. Furthermore, it is understood by those of ordinary skill the art that each nucleotide sequences disclosed herein also encompasses the complement of that exemplified nucleotide sequence.

Further, it is recognized that, for expression of a polynucleotides of the invention in a plant, the polynucleotide is typically operably linked to a promoter that is capable of driving gene expression in the plant of interest. The methods of the invention do not depend on particular promoter. The methods encompass the use of any promoter that is known in the art and that is capable of driving gene expression in the plant of interest.

In certain embodiments, the methods of the present invention involve transforming wheat plants with wheat AHASL1A S653N polynucleotides that are provided in expression cassettes for expression in wheat plants. The cassette will include 5′ and 3′ regulatory sequences operably linked to a wheat AHASL1A S653N polynucleotide. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the wheat AHASL1A S653N polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a the wheat AHASL1A S653N polynucleotide of the invention, and a transcriptional and translational termination region (i.e., termination region) functional in plants. The promoter may be native or analogous, or foreign or heterologous, to the plant host and/or to the wheat AHASL1A S653N polynucleotide. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the wheat AHASL1A S653N polynucleotide, it is intended that the promoter is not the native or naturally occurring promoter for the operably linked wheat AHASL1A S653N polynucleotide. As used herein, a chimeric gene comprises a coding sequence operably linked to a transcription initiation region that is heterologous to the coding sequence.

While it may be preferable to express the wheat AHASL1A S653N polynucleotides using heterologous promoters, the native promoter sequences may be used. Such constructs would change expression levels of the wheat AHASL1A S653N protein in the plant or plant cell. Thus, the phenotype of the plant or plant cell is altered.

The termination region may be native with the transcriptional initiation region, may be native with the operably linked to the wheat AHASL1A S653N polynucleotide, may be native with the plant host, or may be derived from another source (i.e., foreign or heterologous to the promoter, the wheat AHASL1A S653N polynucleotide of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92: 1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Nucleotide sequences for enhancing gene expression can also be used in the plant expression vectors. These include the introns of the maize AdhI, intron1 gene (Callis et al. Genes and Development 1:1183-1200, 1987), and leader sequences, (W-sequence) from the Tobacco Mosaic virus (TMV), Maize Chlorotic Mottle Virus and Alfalfa Mosaic Virus (Gallie et al. Nucleic Acid Res. 15:8693-8711, 1987 and Skuzeski et al. Plant Mol. Biol. 15:65-79, 1990). The first intron from the shrunken-1 locus of maize, has been shown to increase expression of genes in chimeric gene constructs. U.S. Pat. Nos. 5,424,412 and 5,593,874 disclose the use of specific introns in gene expression constructs, and Gallie et al. (Plant Physiol. 106:929-939, 1994) also have shown that introns are useful for regulating gene expression on a tissue specific basis. To further enhance or to optimize AHAS small subunit gene expression, the plant expression vectors of the invention may also contain DNA sequences containing matrix attachment regions (MARs). Plant cells transformed with such modified expression systems, then, may exhibit overexpression or constitutive expression of a nucleotide sequence of the invention.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced AHASL1 expression within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

In one embodiment, the nucleic acids of interest are targeted to the chloroplast for expression. In this manner, where the nucleic acid of interest is not directly inserted into the chloroplast, the expression cassette will additionally contain a chloroplast-targeting sequence comprising a nucleotide sequence that encodes a chloroplast transit peptide to direct the gene product of interest to the chloroplasts. Such transit peptides are known in the art. With respect to chloroplast-targeting sequences, “operably linked” means that the nucleic acid sequence encoding a transit peptide (i.e., the chloroplast-targeting sequence) is linked to the wheat AHASL1A S653N polynucleotide such that the two sequences are contiguous and in the same reading frame. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. While the AHASL1 proteins of the invention include a native chloroplast transit peptide, any chloroplast transit peptide known in art can be fused to the amino acid sequence of a mature AHASL1A protein of the invention by operably linking a chloroplast-targeting sequence to the 5′-end of a nucleotide sequence encoding a mature AHASL1A protein of the invention.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

As disclosed herein, the invention provides methods for producing high protein wheat plants that comprise resistance to an AHAS-inhibiting herbicide. The wheat plants comprise in their genomes at least one copy of a wheat AHASL1A S653N gene. Such a gene may be an endogenous gene or a transgene as disclosed herein. Additionally, in certain embodiments, the wheat AHASL1A S653N gene can be stacked with any combination of polynucleotide sequences of interest, including other herbicide-resistant AHASL1 genes, in order to create wheat plants with a desired phenotype. For example, the polynucleotides of the present invention may be stacked with any other polynucleotides encoding polypeptides having pesticidal and/or insecticidal activity, such as, for example, the Bacillus thuringiensis toxin proteins (described in U.S. Pat. Nos. 5,366,892; 5,747,450; 5,737,514; 5,723,756; 5,593,881; and Geiser et al. (1986) Gene 48:109). The combinations generated can also include multiple copies of any one of the polynucleotides of interest.

The expression cassettes of the invention can include a selectable marker gene for the selection of transformed cells. Selectable marker genes, including those of the present invention, are utilized for the selection of transformed cells or tissues. Marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

The polynucleotide constructs and expression cassettes comprising the wheat AHASL1A S653N polynucleotides can be used in vectors to transform wheat plants. The wheat AHASL1A S653N polynucleotides can be used in vectors alone or in combination with a nucleotide sequence encoding the small subunit of the AHAS (AHASS) enzyme in conferring herbicide resistance in plants. See, U.S. Pat. No. 6,348,643; which is herein incorporated by reference.

The invention also relates to a method for creating a transgenic wheat plant that is produces grain with increased protein content and that is resistant to herbicides, comprising transforming a plant with a polynucleotide construct comprising a promoter that drives expression in a plant operably linked to a wheat AHASL1A S653N polynucleotide.

The invention also relates to the non-transgenic wheat plants, transgenic wheat plants produced by the methods of the invention, and progeny and other descendants of such non-transgenic and transgenic wheat plants, which plants exhibit enhanced or increased resistance to herbicides that interfere with the AHAS enzyme, particularly imidazolinone and sulfonylurea herbicides and produce grain with increased protein content.

The high protein wheat plants of the present invention can comprise in their genomes, in addition to at least one copy of a wheat AHASL1A S653N gene, one or more additional AHASL polynucleotides. Nucleotide sequences encoding herbicide-tolerant AHASL proteins and herbicide-tolerant plants comprising an endogenous gene that encodes a herbicide-tolerant AHASL protein include the polynucleotides and plants of the present invention and those that are known in the art. See, for example, U.S. Pat. Nos. 5,013,659, 5,731,180, 5,767,361, 5,545,822, 5,736,629, 5,773,703, 5,773,704, 5,952,553 and 6,274,796; all of which are herein incorporated by reference.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Cousins, et al. (1991) Aust. J. Plant Physiol. 18:481-494; Chee, P. P. and Slightom, J. L. (1992) Gene 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P: 119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

The methods of the invention involve introducing a polynucleotide construct into a plant. By “introducing a polynucleotide construct” is intended to mean presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the wheat AHASL1A S653N polynucleotides are inserted using standard techniques into any vector known in the art that is suitable for expression of the nucleotide sequences in a plant or plant cell. The selection of the vector depends on the preferred transformation technique and the target plant species to be transformed. In an embodiment of the invention, a wheat AHASL1A S653N polynucleotide is operably linked to a plant promoter that is known for high-level expression in a plant cell, and this construct is then introduced into a plant that that is susceptible to an imidazolinone herbicide and a transformed plant it regenerated. The transformed plant is tolerant to exposure to a level of an imidazolinone herbicide that would kill or significantly injure an untransformed plant. This method can be applied to any plant species; however, it is most beneficial when applied to crop plants, particularly crop plants that are typically grown in the presence of at least one herbicide, particularly an imidazolinone herbicide.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The wheat AHASL1A S653 polynucleotides of the invention may be introduced into plants by contacting plants with a virus or viral nucleic acids. Generally, such methods involve incorporating a polynucleotide construct of the invention within a viral DNA or RNA molecule. It is recognized that the a wheat AHASL1A S653 polynucleotide may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

The high protein wheat plants of the present invention find use in methods for controlling weeds. Thus, the present invention further provides a method for controlling weeds in the vicinity of a high protein wheat plant of the invention. The method comprises applying an effective amount of a herbicide to the weeds and to the high protein wheat plant, wherein the high protein wheat plant has increased resistance to at least one herbicide, particularly an imidazolinone or sulfonylurea herbicide, when compared to a similar, wild-type wheat plant.

By providing high protein wheat plants having increased resistance to herbicides, particularly imidazolinone and sulfonylurea herbicides, a wide variety of formulations can be employed for protecting plants from weeds, so as to enhance plant growth and reduce competition for nutrients. A herbicide can be used by itself for pre-emergence, post-emergence, pre-planting and at planting control of weeds in areas surrounding the plants described herein or an imidazolinone herbicide formulation can be used that contains other additives. The herbicide can also be used as a seed treatment. That is an effective concentration or an effective amount of the herbicide, or a composition comprising an effective concentration or an effective amount of the herbicide can be applied directly to the seeds prior to or during the sowing of the seeds. Additives found in an imidazolinone or sulfonylurea herbicide formulation or composition include other herbicides, detergents, adjuvants, spreading agents, sticking agents, stabilizing agents, or the like. The herbicide formulation can be a wet or dry preparation and can include, but is not limited to, flowable powders, emulsifiable concentrates and liquid concentrates. The herbicide and herbicide formulations can be applied in accordance with conventional methods, for example, by spraying, irrigation, dusting, coating, and the like.

The present invention provides methods for producing a high protein wheat plant, through conventional plant breeding involving sexual reproduction. The methods comprise crossing a first parent wheat plant that comprises in its genome at least one copy of a wheat AHASL1A S653N gene or polynucleotide to a second parent wheat plant so as to produce F1 progeny. The first plant can be any of the high protein wheat plants of the present invention including, for example, transgenic wheat plants comprising at least at least one copy of a wheat AHASL1A S653N gene or and non-transgenic wheat plants that comprise the wheat AHASL1A S653N gene such as those produced by mutagenesis as disclosed in WO 2004/106529 and U.S. Patent Application Publication Nos. 2004/0237134 and 2004/0244080; all of which are herein incorporated by reference. The second parent wheat plant can be any wheat plant that is capable of producing viable progeny wheat plants (i.e., seeds) when crossed with the first plant. Typically, but not necessarily, the first and second parent wheat plants are of the same wheat species. The methods can further involve selfing the F1 progeny to produce F2 progeny. Additionally, the methods of the invention can further involve one or more generations of backcrossing the F1 or F2 progeny plants to a plant of the same line or genotype as either the first or second parent wheat plant. Alternatively, the F1 progeny of the first cross or any subsequent cross can be crossed to a third wheat plant that is of a different line or genotype than either the first or second plant. The methods of the invention can additionally involve selecting plants that comprise the herbicide resistance characteristics of the first plant, for example, by applying an effective amount of a herbicide to the progeny wheat plants that comprise the wheat AHASL1 S653N gene or by standard methods to detect the AHASL1 S653N gene such as, for example, PCR.

The present invention provides methods that involve the use of an AHAS-inhibiting herbicide. In these methods, the AHAS-inhibiting herbicide can be applied by any method known in the art including, but not limited to, seed treatment, soil treatment, and foliar treatment.

Prior to application, the AHAS-inhibiting herbicide can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Examples of suitable carriers are ground natural minerals (for example kaolins, clays, talc, chalk) and ground synthetic minerals (for example highly disperse silica, silicates).

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example Dichlorophen und enzylalkoholhemiformal.

Seed Treatment formulations may additionally comprise binders and optionally colorants.

Binders can be added to improve the adhesion of the active materials on the seeds after treatment. Suitable binders are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethanes, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

An examples of a suitable gelling agent is carrageen (Satiagel™)

Powders, materials for spreading, and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the AHAS-inhibiting herbicide. In this case, the AHAS-inhibiting herbicides are employed in a purity of from 90% to 100% by weight, preferably 95% to 100% by weight (according to NMR spectrum). For seed treatment purposes, respective formulations can be diluted 2-10 fold leading to concentrations in the ready to use preparations of 0.01 to 60% by weight active compound by weight, preferably 0.1 to 40% by weight.

The AHAS-inhibiting herbicide can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the AHAS-inhibiting herbicide according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The AHAS-inhibiting herbicide may also be used successfully in the ultra-low-volume process (ULV), it being possible to apply formulations comprising over 95% by weight of active compound, or even to apply the active compound without additives.

The following are examples of formulations:

1. Products for dilution with water for foliar applications. For seed treatment purposes, such products may be applied to the seed diluted or undiluted.

A) Water-Soluble Concentrates (SL, LS)

Ten parts by weight of the AHAS-inhibiting herbicide are dissolved in 90 parts by weight of water or a water-soluble solvent. As an alternative, wetters or other auxiliaries are added. The AHAS-inhibiting herbicide dissolves upon dilution with water, whereby a formulation with 10% (w/w) of AHAS-inhibiting herbicide is obtained.

B) Dispersible Concentrates (DC)

Twenty parts by weight of the AHAS-inhibiting herbicide are dissolved in 70 parts by weight of cyclohexanone with addition of 10 parts by weight of a dispersant, for example polyvinylpyrrolidone. Dilution with water gives a dispersion, whereby a formulation with 20% (w/w) of AHAS-inhibiting herbicide is obtained.

C) Emulsifiable Concentrates (EC)

Fifteen parts by weight of the AHAS-inhibiting herbicide are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of AHAS-inhibiting herbicide is obtained.

D) Emulsions (EW, EO, ES)

Twenty-five parts by weight of the AHAS-inhibiting herbicide are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of AHAS-inhibiting herbicide is obtained.

E) Suspensions (SC, OD, FS)

In an agitated ball mill, 20 parts by weight of the AHAS-inhibiting herbicide are comminuted with addition of 10 parts by weight of dispersants, wetters and 70 parts by weight of water or of an organic solvent to give a fine AHAS-inhibiting herbicide suspension. Dilution with water gives a stable suspension of the AHAS-inhibiting herbicide, whereby a formulation with 20% (w/w) of AHAS-inhibiting herbicide is obtained.

F) Water-Dispersible Granules and Water-Soluble Granules (WG, SG)

Fifty parts by weight of the AHAS-inhibiting herbicide are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the AHAS-inhibiting herbicide, whereby a formulation with 50% (w/w) of AHAS-inhibiting herbicide is obtained.

G) Water-Dispersible Powders and Water-Soluble Powders (WP, SP, SS, WS)

Seventy-five parts by weight of the AHAS-inhibiting herbicide are ground in a rotor-stator mill with addition of 25 parts by weight of dispersants, wetters and silica gel. Dilution with water gives a stable dispersion or solution of the AHAS-inhibiting herbicide, whereby a formulation with 75% (w/w) of AHAS-inhibiting herbicide is obtained.

I) Gel-Formulation (GF)

In an agitated ball mill, 20 parts by weight of the AHAS-inhibiting herbicide are comminuted with addition of 10 parts by weight of dispersants, 1 part by weight of a gelling agent wetters and 70 parts by weight of water or of an organic solvent to give a fine AHAS-inhibiting herbicide suspension. Dilution with water gives a stable suspension of the AHAS-inhibiting herbicide, whereby a formulation with 20% (w/w) of AHAS-inhibiting herbicide is obtained. This gel formulation is suitable for us as a seed treatment.

2. Products to be applied undiluted for foliar applications. For seed treatment purposes, such products may be applied to the seed diluted.

A) Dustable Powders (DP, DS)

Five parts by weight of the AHAS-inhibiting herbicide are ground finely and mixed intimately with 95 parts by weight of finely divided kaolin. This gives a dustable product having 5% (w/w) of AHAS-inhibiting herbicide.

B) Granules (GR, FG, GG, MG)

One-half part by weight of the AHAS-inhibiting herbicide is ground finely and associated with 95.5 parts by weight of carriers, whereby a formulation with 0.5% (w/w) of AHAS-inhibiting herbicide is obtained. Current methods are extrusion, spray-drying or the fluidized bed. This gives granules to be applied undiluted for foliar use.

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

For seed treatment, seeds of the high protein wheat plants according of the present invention are treated with herbicides, preferably herbicides selected from the group consisting of AHAS-inhibiting herbicides such as amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid, pyrithiobac, and mixtures thereof, or with a formulation comprising a AHAS-inhibiting herbicide. More preferably, the seeds of the high protein wheat plants according of the present invention are treated with an imidazolinone herbicide.

The term seed treatment comprises all suitable seed treatment techniques known in the art, such as seed dressing, seed coating, seed dusting, seed soaking, and seed pelleting.

In accordance with one variant of the present invention, a further subject of the invention is a method of treating soil by the application, in particular into the seed drill: either of a granular formulation containing the AHAS-inhibiting herbicide as a composition/formulation (e.g. a granular formulation, with optionally one or more solid or liquid, agriculturally acceptable carriers and/or optionally with one or more agriculturally acceptable surfactants. This method is advantageously employed, for example, in seedbeds of cereals, maize, cotton, and sunflower.

The present invention also comprises seeds coated with or containing with a seed treatment formulation comprising at least one ALS inhibitor selected from the group consisting of amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac. Preferably, the ALS inhibitor is an imidazolinone herbicide.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

The seed treatment application with the AHAS-inhibiting herbicide or with a formulation comprising the AHAS-inhibiting herbicide is carried out by spraying or dusting the seeds before sowing of the plants and before emergence of the plants.

In the treatment of seeds, the corresponding formulations are applied by treating the seeds with an effective amount of the AHAS-inhibiting herbicide or a formulation comprising the AHAS-inhibiting herbicide. Herein, the application rates are generally from 0.1 g to 10 kg of the a.i. (or of the mixture of a.i. or of the formulation) per 100 kg of seed, preferably from 1 g to 5 kg per 100 kg of seed, in particular from 1 g to 2.5 kg per 100 kg of seed. For specific crops such as lettuce the rate can be higher.

The high protein wheat plant of the present invention find use in a method for combating undesired vegetation or controlling weeds comprising contacting the seeds of the high protein wheat plants according to the present invention before sowing and/or after pregermination with an AHAS-inhibiting herbicide. The method can further comprise sowing the seeds, for example, in soil in a field or in a potting medium in greenhouse. The method finds particular use in combating undesired vegetation or controlling weeds in the immediate vicinity of the seed.

The control of undesired vegetation is understood as meaning the killing of weeds and/or otherwise retarding or inhibiting the normal growth of the weeds. Weeds, in the broadest sense, are understood as meaning all those plants which grow in locations where they are undesired.

The weeds of the present invention include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindemia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

In addition, the weeds of the present invention can include, for example, crop plants that are growing in an undesired location. For example, a volunteer maize plant that is in a field that predominantly comprises soybean plants can be considered a weed, if the maize plant is undesired in the field of soybean plants.

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The following examples are offered by way of illustration and not by way of limitation.

### Example 1

### Wheat Lines with Increased Grain Protein Content

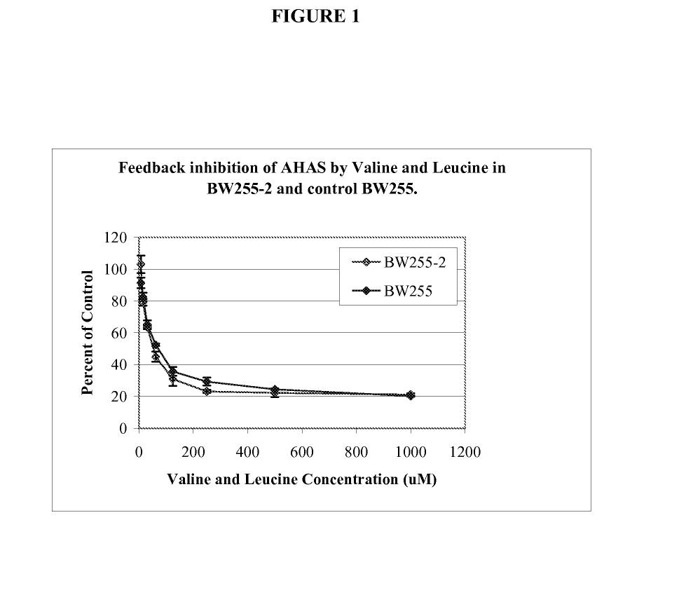

Wheat lines were produced using standard mutagenesis and conventional plant breeding methods. The objective of the mutagenesis was to develop wheat lines with tolerance to imidazolinones herbicides. The mutation responsible for imidazolinone tolerance in these wheat lines is a single nucleotide change of guanine to adenine, which results in a codon change from AGC to AAC and a single amino acid substitution of serine to asparagine in the AHASL (acetohydroxyacid synthase large subunit) protein, designated as TaAHASL1A S653N. The AHAS enzyme catalyzes the first step in the biosynthesis of branched-chain amino acids, valine, leucine and isoleucine (Stidham and Singh (1991) “Imidazolinone-Acetohydroxyacid Synthase Interactions,” In: The Imidazolinone Herbicides, Ch. 6, Shaner, D., and O'Connor, S., eds.; CRC Press, Boca Raton, Fla., U.S.A., pp. 71-90) and is under feedback regulation by these amino acids in plants. The single point mutation in the AHAS gene confers tolerance to imidazolinone herbicides by altering the binding site for these herbicides on the mutant AHAS enzyme, but has no recognized effect on feedback regulation by branched-chain amino acids and the normal biosynthetic function of the enzyme (Newhouse et al., (1992) Plant Physiol. 100:882-886) (FIG. 1).

Grain compositional studies were conducted during the selection process of wheat lines exhibiting herbicide tolerance and from these studies the higher grain protein content was discovered. These studies were conducted in different geographical locations (California, Minnesota, North Dakota, Washington State and Canada) and years from 1999 until 2004 (Table 2). The five lines (BW255-2, BW238-3, K42, Teal15A and ElsaxEM2) exhibiting this trait are independently derived from different germplasm, and by independent mutagenesis events, and one line (ElsaxEM2) was derived through introgression from Einkorn wheat (Triticum monococcum) which had been mutagenized.

Percent protein values were higher for lines BW255-2, BW238-3, K42, Teal15A and ElsaxEM2 as compared to their parents (Table 1). The significant increases for years and locations ranged from 3 to 13% as compared to their respective parental line (Table 2) or an actual increase from 0.4 to 2.1%, averaging 1.3% across all lines locations and years as compared to their parents. Values for the branched change amino acids valine, isoleucine and leucine and essential amino acids lysine, methionine, cystine and threonine were usually significantly higher but there were some exceptions (Table 2). The average increase ranged from 6 to 11% as compared to their respective parental line (Table 2) or an actual increase from 0.02 to 0.09 averaging 0.04% across all amino acid values compared to their parents. Grain yield and test weigh values for mutants BW255-2 and BW238-3 were not significantly different than their respective parental lines for field trials grown in 2003 and 2004 (Table 3). Likewise, the feedback inhibition results presented for lines BW255-2 and parental BW255 (FIG. 1) are comparable for the other lines and shows that there was no effect of the mutation on feedback inhibition which could have altered the regulation of the branched chain amino acids biosynthesis.

The herbicide tolerant wheat lines used in the studies presented in this example are generation M5 or greater and are homozygous for the AHASL1A S653N trait.

TABLE 1
Average percent increase in grain protein content for
lines homozygous for AHASL1A S653N as compared to their
parents summarized across locations and years.

| | Protein |
|---|---|
| Lines | (% Increase) |
| BW238-3 | 7.4 |
| BW255-2 | 7.3 |
| K42 | 13.3 |
| EM2 × Elsa | 5.6 |
| Teal15A | 8.2 |

TABLE 2
Comparison of protein and amino acid values of TaAHASLlA
S653N mutants to their parental backgrounds.

| Year | Analyte % | Teal^{1,2} | Teal15A | % difference | | | |
|---|---|---|---|---|---|---|---|
| 1999 | Protein | 15.9 b | 17.2 a | 8.2 | | | |
| | Valine | 0.71 b | 0.75 a | 5.6 | | | |
| | Isoleucine | 0.57 b | 0.62 a | 8.8 | | | |
| | Leucine | 1.06 b | 1.15 a | 8.5 | | | |
| | Lysine | 0.41 b | 0.44 a | 7.3 | | | |
| | Methionine | 0.25 a | 0.27 a | 8.0 | | | |
| | Cystine | 0.37 a | 0.39 a | 5.4 | | | |
| | Threonine | 0.52 b | 0.56 a | 7.7 | | | |
| Year | Analyte % | BW238 | BW238-3 | % difference | BW255 | BW255-2 | % difference |
| 2002 | Protein | 17.5 a | 19.1 bc | 9.1 | 18.3 ab | 20.4 c | 11.5 |
| | Valine | 0.7 a | 0.77 bc | 10.0 | 0.75 b | 0.81 c | 8.0 |
| | Isoleucine | 0.53 a | 0.67 b | 26.4 | 0.63 b | 0.69 b | 9.5 |
| | Leucine | 1.05 a | 1.26 b | 20.0 | 1.21 b | 1.32 b | 9.1 |
| | Lysine | 0.44 a | 0.48 bc | 9.1 | 0.47 b | 0.5 c | 6.4 |
| | Methionine | 0.27 a | 0.3 b | 11.1 | 0.3 b | 0.33 c | 10.0 |
| | Cystine | 0.35 a | 0.39 b | 11.4 | 0.37 ab | 0.41 c | 10.8 |
| | Threonine | 0.51 a | 0.56 c | 9.8 | 0.53 b | 0.59 d | 11.3 |
| Year | Analyte % | BW238 | BW238-3 | % difference | BW255 | BW255-2 | % difference |
| 2003 | Protein | 16.9 a | 18.1 bc | 7.1 | 18 ab | 19.4 c | 7.8 |
| | Valine | 0.72 a | 0.78 b | 8.3 | 0.73 a | 0.81 b | 11.0 |
| | Isoleucine | 0.6 a | 0.65 b | 8.3 | 0.62 ab | 0.7 c | 12.9 |
| | Leucine | 1.17 a | 1.26 b | 7.7 | 1.21 ab | 1.33 c | 9.9 |
| | Lysine | 0.44 a | 0.46 b | 4.5 | 0.43 a | 0.47 b | 9.3 |
| | Methionine | 0.25 a | 0.27 b | 8.0 | 0.25 a | 0.28 b | 12.0 |
| | Cystine | 0.35 a | 0.36 a | 2.9 | 0.36 a | 0.41 b | 13.9 |
| | Threonine | 0.52 a | 0.57 bc | 9.6 | 0.55 ab | 0.58 c | 5.5 |
| | Analyte % | Elsa | EM2xElsa | % difference | Krichuaff | K42 | % difference |
| | Protein | 16.2 b | 17.1 a | 5.6 | 13.5 b | 15.3 a | 13.3 |
| | Valine | 0.71 b | 0.75 a | 5.6 | 0.59 b | 0.71 a | 20.3 |
| | Isoleucine | 0.58 b | 0.62 a | 6.9 | 0.46 b | 0.57 a | 23.9 |
| | Leucine | 1.18 b | 1.11 a | −5.9 | 0.93 b | 1.06 a | 14.0 |
| | Lysine | 0.42 b | 0.44 a | 4.8 | 0.38 b | 0.42 a | 10.5 |
| | Methionine | 0.24 a | 0.25 a | 4.2 | 0.22 a | 0.23 a | 4.5 |
| | Cystine | 0.36 a | 0.37 a | 2.8 | 0.35 a | 0.36 a | 2.9 |
| | Threonine | 0.5 b | 0.53 a | 6.0 | 0.46 a | 0.5 a | 8.7 |
| Year | Analyte % | BW238 | BW238-3 | % difference | BW255 | BW255-2 | % difference |
| 2004 | Protein | 16.7 a | 17.7 b | 6.0 | 15.2 c | 15.6 d | 2.6 |
| | Valine | 0.69 e | 0.66 cd | −4.3 | 0.62 a | 0.65 bc | 4.8 |
| | Isoleucine | 0.56 e | 0.53 d | −5.4 | 0.49 b | 0.52 cd | 6.1 |
| | Leucine | 1.14 a | 1.18 b | 3.5 | 1.03 c | 1.08 d | 4.9 |
| | Lysine | 0.42 b | 0.42 b | 0.0 | 0.39 a | 0.41 b | 5.1 |
| | Methionine | 0.23 bc | 0.24 cd | 4.3 | 0.21 a | 0.24 cd | 14.3 |
| | Cystine | 0.33 c | 0.37 d | 12.1 | 0.29 a | 0.32 bc | 10.3 |
| | Threonine | 0.47 c | 0.5 d | 6.4 | 0.44 a | 0.46 b | 4.5 |

¹Values are the means of nine observations (% dry weight basis). Mutants and parental sources were grown in replicated block field trials.
²Statistical analysis was done within a given analyte comparing mutant to parental. Like letters are not significantly different.

TABLE 3
Yield and test weight values of parental lines BW255
and BW238 and mutants BW255-2 and BW238-3 (TaAHASL1A S653N)
grown in three locations in the U.S during 2003 and 2004.

| | | Grain | | Test | |
|---|---|---|---|---|---|
| | | Yield | | Weight | |
| Year | Variety | bu/A)¹ | Group*² | (lbs/bu) | Group* |
| 2003 | BW255 | 60.9 | a | 60.1 | a |
| | BW255-2 | 60.6 | a | 60 | a |
| | BW238 | 60.4 | a | 59.4 | a |
| | BW238-3 | 60.6 | a | 61.6 | a |
| | | F = 0.57 | LSD = 1.0 | F = 0.85 | LSD = 3.0 |
| | | P 0.64 | | P 0.45 | |
| | | Grain | | Test | |
| | | Yield | | Weight | |
| Year | Variety | bu/A) | Group* | (lbs/bu) | Group* |
| 2004 | BW255 | 57.4 | ab | 61.9 | ab |
| | BW255-2 | 54.4 | a | 62.4 | b |
| | BW238 | 65..4 | b | 59.6 | a |
| | BW238-3 | 59.1 | ab | 59.3 | a |
| | | F = 1.86 | LSD = 9.8 | F = 2.00 | LSD = 2.7 |
| | | P = 0.149 | | P = 0.151 | |

¹Values are the means of nine observations grown in randomized complete block designs from field sites in ND and MN.
²Like letters are not significantly different.

Grain protein content, branched chain and essential amino acids values from bread wheat lines that are resistant to imidazolinones herbicide were significantly increased as compared to their respective parental lines. The four independently derived lines having the Triticum aestivum AHASL1A S653N gene and another derived through introgression of the same mutation from Triticum monococcum L. all exhibited the increase in grain protein trait, when compared to their respective parent lines. These results demonstrate that the increase in grain protein is due to the wheat AHASL1A S653N mutation and that there was neither a decrease in grain yield nor a change in the feedback inhibition response in these AHASL1A S653N lines as compared to the parents. While all of the AHASL1A S653N wheat lines examined thus far comprise the AAC codon for the asparagine 653, wheat lines comprising an AAT codon for the asparagine 653 are also expected to produce grain with increased protein content.

The advantage of increased grain protein content provided by the S653N mutation is limited only to the AHASL1A gene. Wheat lines with the S653N mutation occurring on homologous AHASL1D and AHASL1B genes did not exhibit the increase in grain protein (data not shown).

### Example 2

### Herbicide-Resistant Wheat AHASL Proteins

The present invention discloses the use of the polynucleotides encoding wheat AHASL1A S653N polypeptides. Plants comprising herbicide-resistant AHASL polypeptides have been previously identified, and a number of conserved regions of AHASL polypeptides that are the sites of amino acid substitutions that confer herbicide resistance have been described. See, Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam; and Devine and Shukla, (2000) Crop Protection 19:881-889.

Using the wheat AHASL1A S653N sequences of the invention and methods known to those of ordinary skill in art, one can produce additional polynucleotides encoding herbicide-resistant AHASL polypeptides having the S653N substitution and one, two, three, or more additional amino acid substitutions at the identified sites in these conserved regions. Table 4 provides the conserved regions of AHASL proteins, the amino acid substitutions known to confer herbicide resistance within these conserved regions, and the corresponding amino acids in the wheat (Triticum aestivum) AHASL1 proteins.

TABLE 4
Amino Acid Substitutions in Conserved Regions of AHASL Polypeptides
that are Known to Confer Herbicide-Resistance.

| | | | Amino acid position in |
|---|---|---|---|
| Conserved region¹ | Mutation² | Reference | Triticum aestivum |
| VFAYPGGASMEIHQALTRS³ | Ala₁₂₂ to Thr | Bernasconi et al.⁴ | Ala₄₈ |
| | | Wright & Penner⁵ | |
| AITGQVPRRMIGT³ | Pro₁₉₇ to Ala | Boutsalis et al.⁶ | Pro₁₂₃ |
| | Pro₁₉₇ to Thr | Guttieri et al.⁷ | |
| | Pro₁₉₇ to His | Guttieri et al.⁸ | |
| | Pro₁₉₇ to Leu | Guttieri et al.⁷ | |
| | | Kolkman et al.⁹ | |
| | Pro₁₉₇ to Arg | Guttieri et al.⁷ | |
| | Pro₁₉₇ to Ile | Boutsalis et al.⁶ | |
| | Pro₁₉₇ to Gln | Guttieri et al.⁷ | |
| | Pro₁₉₇ to Ser | Guttieri et al.⁷ | |
| AFOETP³ | Ala₂₀₅ to Asp | Hartnett et al.¹⁰ | Ala₁₃₁ |
| | Ala₂₀₅ to Val¹¹ | Simpson¹¹ | |
| | | Kolkman et al.⁹ | |
| | | White et al.¹² | |
| QWED³ | Trp₅₇₄ to Leu | Bruniard¹³ | Trp₅₀₀ |
| | | Boutsalis et al.⁶ | |
| IPSGG³ | Ser₆₅₃ to Asn | Devine & Eberlein¹⁴ | Ser₅₇₉ |
| | | Chang & Duggleby¹⁵ | |
| | Ser₆₅₃ to Thr | Lee et al.¹⁶ | |
| | Ser₆₅₃ to Phe | | |

¹Conserved regions from Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam and Devine and Shukla, (2000) Crop Protection 19: 881-889.
²Amino acid numbering corresponds to the amino acid sequence of the Arabidopsis thaliana AHASL polypeptide.
³The amino acid sequence of the wild-type Triticum aestivum AHASL1 comprises the same conserved region.
⁴Bernasconi et al. (1995) J. Biol. Chem. 270(29): 17381-17385.
⁵Wright and Penner (1998) Theor. Appl. Genet. 96: 612-620.
⁶Boutsalis et al. (1999) Pestic. Sci. 55: 507-516.
⁷Guttieri et al. (1995) Weed Sci. 43: 143-178.
⁸Guttieri et al. (1992) Weed Sci. 40: 670-678.
⁹Kolkman et al. (2004) Theor. Appl. Genet. 109: 1147-1159.
¹⁰Hartnett et al. (1990) “Herbicide-resistant plants carrying mutated acetolactate synthase genes,” In: Managing Resistance to Agrochemicals: Fundamental Research to Practical Strategies, Green et al. (eds.), American Chemical Soc. Symp., Series No. 421, Washington, DC, USA
¹¹Simpson (1998) Down to Earth 53(1): 26-35.
¹²White et al. (2003) Weed Sci. 51: 845-853.
¹³Bruniard (2001) Inheritance of imidazolinone resistance, characterization of cross-resistance pattern, and identification of molecular markers in sunflower (Helianthus annuus L.). Ph.D. Thesis, North Dakota State University, Fargo, ND, USA, pp 1-78.
¹⁴Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam.
¹⁵Chang and Duggleby (1998) Biochem J. 333: 765-777.
¹⁶Lee et al. (1999) FEBS Lett. 452: 341-345.

### Example 3

### Performance of High Protein Wheat Lines in Arizona and California Field Trials

Spring wheat lines (Triticum aestivum) comprising the AHASL1A S653(At)N mutation and their isogenic, non-mutant, parental lines were grown over the winter (2005-2006) in three locations in the Northern Hemisphere (California and Arizona, USA). The grain protein content of each of the lines was measured to determine whether the AHASL1A S653N mutant wheat lines displayed increased grain grown relative to their parental lines in environments that are outside of their adaptation zones and under sub-optimal photoperiod conditions (i.e., shorter days).

### Entries and Locations

Homozygous AHASL1A (S653N) mutants in two genetically distinct genotypes, Kirchauff-K42 (an Australian spring wheat line, also referred to herein as “K42”) and BW238-3 (a North American spring wheat line), along with their isogenic, non-mutant, parental lines (Kirchauff and BW238 respectively) were grown in adjacent large plots (single repetition) at three locations over the 2005-2006 winter season in the southwestern United States. Two locations were close to Yuma, Ariz. while the third location was in the vicinity of Dinuba, Calif. The locations were planted in November 2005 and harvested in July 2006.

### Plot Dimensions and Seeding Rates

Seeding Rate: 100 g seed per 35 m².

Plot Size: 2×1.75 m×10 m (1 Rep.).

Plots were separated by 10 m wide barley strips.

### Agronomic Performance and Grain Harvest

All plots were subjected to the same agronomic practices. None of the plots were treated with imidazolinone herbicides. To demonstrate the genotypic distinctiveness of the Kirchauff and BW238 lines, plots were evaluated for growth habit and height. The Kirchauff-K42 line and its isogenic parental line, Kirchauff, grew taller and exhibited less tillering than the BW238-3 line and its isogenic parental line, BW238. No significant differences in agronomic performance were detected between the lines containing the AHASL1A S653N mutation and their respective isogenic non-mutant parental lines when observed in the field at each of the locations. Table 5 provides a summary of the growth habits of all four lines at the Dinuba, Calif. location.

TABLE 5
Growth characteristics of four bread wheat lines
grown in the winter season in Dinuba, California.

| | Evaluation on Jan. 25, 2006 | | | Evaluation on Feb. 10, 2006 | | |
|---|---|---|---|---|---|---|
| | Plant Height | Growth | | Plant Height | Growth | |
| Wheat Line | (cm) | Stage | Remarks | (cm) | Stage | Remarks |
| BW-238-3 | 10-15 | 24-27 | Very | 23-30 | 25-30 | Heavy tillering. |
| (S653N line) | | | prostrate | | | End of tillering |
| BW-238 | 10-14 | 24-27 | growth | 23-30 | 25-30 | to becoming |
| (parental line) | | | | | | erect. |
| K42 | 20-33 | 24-30 | Erect | 38-52 | 31 | Moderately |
| (S653N line) | | | growth | | | tillered. |
| Kirchauff | 20-33 | 24-30 | | 38-52 | 31 | Completely erect. |
| (parental line) | | | | | | at 1^{st} node stage. |

### Results and Discussion

The grain test weights, SDS sedimentation values, and percent protein content from the two Yuma, Ariz. locations and the Dinuba, Calif. location are provided in Table 6-8, respectively. Table 9 provides a summary of the results across all three locations. When the grain protein content results were averaged across the three locations, Kirchauff-K42 displayed a level of grain protein that was 5% higher than its isogenic parental control line (Table 9). Similarly, BW238-3 displayed a level of grain protein that was 5.1% higher than its isogenic parental control line when grain protein content was averaged across the three locations (Table 9). The average grain test weight was slightly higher for the Kirchauff-K42 compared to its non-mutant parental line; whereas the grain test weight of the BW238-3 was not significantly different from its non-mutant parental line (Table 9). The SDS sedimentation values, which are used to predict gluten strength and baking quality were also not significantly different between the mutant AHASL1A lines and the respective parental controls.

These results demonstrate that hexaploid bread wheat lines containing the AHASL1A S653N mutation produce grain with a higher in grain protein content than parental control lines even when grown outside of their adaptation zones and outside of their normal growing season.

TABLE 6
Grain test weights (lbs/bu), % grain protein content,
and SDS sedimentation (mm) values of TaAHASL1A S653N
mutant lines and parental lines in Yuma Trial 1.

| | | SDS | Grain | Percent |
|---|---|---|---|---|
| | | Sedimen- | Protein | Increase in |
| | Test Weight | tation* | Content | Grain Protein |
| Line | (lbs/bu) | (mm) | (%) | Content^{†} |
| K42 | 61.4 | 99 | 14.4 | 3.6 |
| (S653N line) | | | | |
| Kirchauff | 57.5 | 99 | 13.9 | — |
| (parental line) | | | | |
| BW 238-3 | 59.1 | 105 | 18.7 | 6.3 |
| (S653N line) | | | | |
| BW238 | 59.5 | 110 | 17.6 | — |
| (parental line) | | | | |

*SDS (Sodium Dodecyl Sulfate) Sedimentation test for wheat is an American Associate of Cereal Chemists (AACC) International Approved Method to predict gluten strength and baking quality in both durum and bread wheats. See, Morris et al. (2007) J. Sci. Food Agric. 87: 607-615.
^{†}Percent increase in grain protein content of S653N line over the grain protein content of the parental line. Kirchauff and BW238 are the parental lines for K42 and BW238-3, respectively.

TABLE 7
Grain test weights (lbs/bu), % grain protein content,
and SDS sedimentation (mm) values of TaAHASL1A S653N
mutant lines and parental lines in Yuma Trial 2.

| | | SDS | Grain | Percent |
|---|---|---|---|---|
| | | Sedimen- | Protein | Increase in |
| | Test Weight | tation | Content | Grain Protein |
| Line | (lbs/bu) | (mm) | (%) | Content |
| K42 | 60.0 | 104 | 14.7 | 4.3 |
| (S653N line) | | | | |
| Kirchauff | 57.6 | 109 | 14.1 | — |
| (parental line) | | | | |
| BW 238-3 | 58.6 | 111 | 19.2 | 5.5 |
| (S653N line) | | | | |
| BW238 | 58.4 | 112 | 18.2 | — |
| (parental line) | | | | |

TABLE 8
Grain test weights (lbs/bu), % grain protein content,
and SDS sedimentation (mm) values of TaAHASL1A S653N
mutant lines and parental lines in Dinuba trial.

| | | SDS | Grain | Percent |
|---|---|---|---|---|
| | | Sedimen- | Protein | Increase in |
| | Test Weight | tation | Content | Grain Protein |
| Line | (lbs/bu) | (mm) | (%) | Content |
| K42 | 62.0 | 99 | 14.7 | 8.1 |
| (S653N line) | | | | |
| Kirchauff | 57.2 | 91 | 13.6 | — |
| (parental line) | | | | |
| BW 238-3 | 59.3 | 114 | 18.1 | 2.3 |
| (S653N line) | | | | |
| BW238 | 58.2 | 116 | 17.7 | — |
| (parental line) | | | | |

TABLE 9
Averages* of grain test weights (lbs/bu), % grain protein
content, and SDS sedimentation (mm) values of TaAHASL1A
S653N mutant lines and parental lines across locations.

| | | | | | | | Percent |
|---|---|---|---|---|---|---|---|
| | Average Test | | Average SDS | | Average Grain | | Increase |
| | Weight | | Sedimentation | | Protein Content | | in Grain |
| | (lbs/bu) | | (mm) | | (%) | | Protein |
| Line | Avg. | s.d.^{†} | Avg. | s.d. | Avg. | s.d. | Content^{@} |
| K42 | 61.1 | 1.0 | 100.7 | 0.2 | 14.6 | 2.9 | 5.0 |
| (S653N | | | | | | | |
| line) | | | | | | | |
| Kirchauff | 57.4 | 0.2 | 99.7 | 0.2 | 13.9 | 9.0 | — |
| (parental | | | | | | | |
| line) | | | | | | | |
| BW 238-3 | 59.0 | 0.4 | 110.0 | 0.3 | 18.7 | 4.6 | 5.1 |
| (S653N | | | | | | | |
| line) | | | | | | | |
| BW238 | 58.7 | 0.7 | 112.7 | 0.1 | 17.8 | 3.1 | — |
| (parental | | | | | | | |
| line) | | | | | | | |

*Average of values from the two Yuma trials (Tables 6 and 7) and the Dinuba Trial (Table 8).
^{†}Standard deviation (s.d.).
^{@}Percent increase in average grain protein content of S653N line over the average grain protein content of the parental line. Kirchauff and BW238 are the parental lines for K42 and BW238-3, respectively.

### Example 4

### Baking Quality Tests of Grain Produced from High Protein Wheat Lines

Samples of grain grown in two of the three locations (one in Dinuba, Calif. and one in Yuma, Ariz.) in the field trials disclosed in Example 3 above were subjected to a number of wheat and flour testing methods by an independent laboratory to determine whether the increase in grain protein in the AHASL1A mutants had an effect on baking quality. Grain samples from each entry (AHASL1A and parental isogenic line) were subjected to a laboratory milling process (Buhler Laboratory Flour Mill) to produce ground wheat and flour samples. Wheat and milled samples were then subjected to a number of quality tests (moisture content, protein content, ash content and falling number) to determine a number of standard wheat quality parameters. Specialized standard tests, such as the Single Kernel Characterization System (SKCS), Farinograph, and Pan Bread bake test were conducted to determine processing and baking characteristics of each sample. These methods are described in “Wheat and Flour Testing Methods. A Guide to Understanding Wheat and Flour Quality”, (2004) Wheat Marketing Center, Inc. and North American Export Grain Association, Inc., USA; herein incorporated by reference. The results of these tests are provided in Tables 10-13 below.

Although the AHASL1A S653N mutant lines all demonstrated an increase in grain protein, none of the mutant lines differed significantly from their parental isogenic lines in terms of bake data (Tables 10-13). This was expected since the SDS sedimentation values, which are used to predict gluten strength and baking quality (see, Example 3 above), were also not significantly different between the mutant AHASL1A lines and the respective parental checks.

To be able to increase grain protein without affecting baking quality is a desirable characteristic for the wheat industry. Thus, the mutant AHASL1A lines of the present invention find use in the production of flour that has increased protein content while maintaining the baking quality of flour from control wheat lines. Flour from grain of the mutant AHASL1A wheat lines also finds use in the production of baked goods with increased protein content, when compared to baked goods produced from flour milled from grain of control or wild-type wheat lines.

TABLE 10
Wheat data.

| Sample | | | Wheat Data | | | | | | |
|---|---|---|---|---|---|---|---|---|---|
| Variety | CMDTY | LOC | PRO | MOI | TW | TKW | HARD | FN | SKCS |
| K42 | HWS | Yuma | 13.48 | 8.67 | 62.6 | 31.82 | 76.03 | 524 | Hard 0-4-7-89-1 |
| Kirchauff | HWS | Yuma | 12.92 | 8.61 | 59.7 | 27.61 | 85.63 | 524 | Hard 1-3-7-89-1 |
| K42 | HWS | Dinuba | 14.09 | 9.1 | 62.8 | 31.14 | 71.61 | 541 | Hard 0-5-19-76-1 |
| Kirchauff | HWS | Dinuba | 13.08 | 8.98 | 59.5 | 26.18 | 81.31 | 567 | Hard 1-2-6-91-1 |
| BW 238-03 | HRS | Yuma | 16.85 | 8.59 | 60.9 | 27.88 | 85.48 | 593 | Hard 0-2-3-95-1 |
| BW 238 | HRS | Yuma | 16.39 | 8.64 | 59.4 | 27.82 | 84.45 | 566 | Hard 0-0-5-95-1 |
| BW 238-03 | HRS | Dinuba | 16.76 | 8.5 | 60.5 | 25.47 | 85.92 | 618 | Hard 0-1-5-94-1 |
| BW 238 | HRS | Dinuba | 16.19 | 8.86 | 60.2 | 24.66 | 85.28 | 604 | Hard 2-2-7-89-1 |

CMDTY, commodity; HWS, hard white spring wheat; and HRS, hard red spring wheat.
LOC, location.
PRO, % protein in wheat at 8.5% moisture.
MOI, moisture (%).
TW, test weight.
TKW, thousand kernel weight (grams).
Hard, Kernel Hardness (index from −20 to 120).
FN, Falling Number (seconds). FN is a measure of viscosity determined by measuring the resistance of a flour and water paste to a falling stirrer.
SKCS, Single Kernel Characterization System. This system analyzes 300 kernels individually for kernel weight (mg), kernel diameter (mm), moisture content (%) and kernel hardness (an index from −20 to 120).

TABLE 11
Flour data and farinograph results.

| Sample | | | Flour Data | | | Farinograph | | | |
|---|---|---|---|---|---|---|---|---|---|
| Variety | CMDTY | LOC | PRO | MOI | ASH | ABS | Peak | Stability | MTI |
| K42 | HWS | Yuma | 11.86 | 13.73 | 0.499 | 63.7 | 6.00 | 9.00 | 30 |
| Kirchauff | HWS | Yuma | 11.61 | 13.5 | 0.511 | 64.0 | 5.00 | 9.00 | 30 |
| K42 | HWS | Dinuba | 12.56 | 12.99 | 0.515 | 64.3 | 5.75 | 6.25 | 45 |
| Kirchauff | HWS | Dinuba | 11.5 | 13.01 | 0.549 | 62.5 | 5.00 | 5.75 | 55 |
| BW 238-03 | HRS | Yuma | 15.72 | 13.35 | 0.605 | 67.8 | 7.00 | 10.25 | 20 |
| BW 238 | HRS | Yuma | 15.43 | 13.77 | 0.551 | 66.4 | 7.50 | 13.75 | 15 |
| BW 238-03 | HRS | Dinuba | 16.01 | 13.52 | 0.564 | 68.0 | 7.75 | 24.50 | 15 |
| BW 238 | HRS | Dinuba | 15.43 | 13.43 | 0.557 | 65.9 | 8.00 | 26.65 | 15 |

CMDTY, commodity; HWS, hard white spring wheat; and HRS, hard red spring wheat.
LOC, location.
PRO, % protein in flour at 14% moisture.
MOI, moisture (%).
ASH, flour ash (%).
ABS, Absorption (%): the amount of water required to center the farinograph curve on the 500-Brabender-Unit (BU) line.
Peak, Peak time (minutes): indicates dough development time, beginning the moment water is added until the dough reaches maximum consistency.
Stability, Stability time (minutes): is the time the dough maintains maximum consistency.
MTI, Mixing Tolerance Index (minutes): indicates the degree of softening of the dough during mixing.

TABLE 12
Bake data.

| Sample | | | Bake Data | | | | | | | | | | |
|---|---|---|---|---|---|---|---|---|---|---|---|---|---|
| Variety | CMDTY | LOC | Vol cc | Vol | Grain | Texture | Color | ABS | Makeup | Hand | Tol | BS | SS |
| K42 | HWS | Yuma | 877 | 10 | 10 | 10 | 0 | 3 | 10 | 5 | 5 | 53 | 55 |
| Kirchauff | HWS | Yuma | 795 | 5 | 10 | 10 | 0 | 3 | 5 | 5 | 5 | 43 | 40 |
| K42 | HWS | Dinuba | 840 | 5 | 5 | 10 | 5 | 3 | 10 | 5 | 5 | 48 | 55 |
| Kirchauff | HWS | Dinuba | 797 | 5 | 10 | 10 | 0 | 3 | 5 | 5 | 5 | 40 | 40 |
| BW 238-03 | HRS | Yuma | 945 | 10 | 5 | 10 | 5 | 3 | 10 | 10 | 5 | 58 | 60 |
| BW 238 | HRS | Yuma | 945 | 10 | 10 | 10 | 5 | 3 | 10 | 10 | 10 | 68 | 60 |
| BW 238-03 | HRS | Dinuba | 955 | 10 | 10 | 10 | 5 | 3 | 10 | 10 | 10 | 68 | 65 |
| BW 238 | HRS | Dinuba | 975 | 10 | 10 | 10 | 5 | 3 | 10 | 10 | 10 | 68 | 65 |

CMDTY, commodity; HWS, hard white spring wheat; and HRS, hard red spring wheat.
LOC, location.
Vol cc, Volume of the baked pan bread (cubic centimeters).
Vol, Specific volume is the ratio of volume to weight
Grain, Pan bread is scored for internal uniform crumb grain.
Texture, Pan bread is scored for texture.
Color, Flour color is determined by measuring the whiteness of a flour sample with the Minolta Chroma Meter and compared to a scale.
ABS, Absorption

TABLE 13
Comments on baking quality tests.

| Sample | | | |
|---|---|---|---|
| Variety | CMDTY | LOC | Comments |
| K42 | HWS | Yuma | Improved protein, TW, TKW, and Bake (still poor bake); |
| | | | Poor yellow color for a ww |
| Kirchauff | HWS | Yuma | Poor bake quality, slightly low TKW, lower protein; Poor |
| | | | yellow color for a ww |
| K42 | HWS | Dinuba | Improved protein, TW, TKW, and Bake (still poor bake |
| | | | though); color slightly better |
| Kirchauff | HWS | Dinuba | Poor bake quality, low TKW; Poor yellow color and very |
| | | | weak dough characteristics |
| BW 238-03 | HRS | Yuma | Very high protein, slightly low TKW, High water |
| | | | absorption, marginal bake |
| BW 238 | HRS | Yuma | High protein, slightly low TWK, High water absorption, |
| | | | Good bake and improved stability |
| BW 238-03 | HRS | Dinuba | High protein, low TKW, Long Stability and High water |
| | | | absorption, Good bake and strong doughs |
| BW238 | HRS | Dinuba | High protein, low TKW, Long Stability, Good bake and |
| | | | strong doughs; similar to 4A |

CMDTY, commodity;
HWS, hard white spring wheat; and
HRS, hard red spring wheat.
LOC, location.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for producing a high protein wheat plant, said method comprising the steps of:
(a) introducing into a wheat plant at least one copy of a wheat AHASL1A S653N gene;
(b) growing the wheat plant or a descendent plant thereof comprising the AHASL1A S653N gene to produce grain; and
(c) determining the protein content of grain produced by the wheat plant or the descendent plant, wherein the wheat plant or the descendent plant produces grain having an increased level of protein when compared to grain produced by a wheat plant lacking said wheat AHASL1A S653N gene.

2. The method of claim 1, wherein wheat AHASL1A S653N gene encodes and AHASL1A protein comprising an asparagine at amino acid position 579 or equivalent position.

3. The method of claim 1, wherein said wheat AHASL1A S653N gene is a Triticum aestivum or Triticum monococcum AHASL1A S653N gene.

4. The method of claim 1, further comprising the step of selecting for a wheat plant comprising said wheat AHASL1A S653N gene.

5. The method of claim 4, wherein said selecting step comprises applying an AHAS-inhibiting herbicide to said wheat plant after said wheat AHASL1A S653N gene is introduced.

6. The method of claim 1, wherein said wheat AHASL1A S653N gene is introduced into said high protein wheat plant by cross pollination.

7. The method of claim 6, wherein said cross pollination comprises crossing a first parent wheat plant to a second parent wheat plant so as to produce at least one F1 progeny, wherein said first parent wheat plant comprises at least one copy of said AHASL1A S653N gene and wherein said high protein wheat plant is descended from said first and said second parent wheat plants.

8. The method of claim 7, wherein said first parent wheat plant is selected from the group consisting of:
(a) a wheat plant having American Type Culture Collection (ATCC) Patent Deposit Designation Number PTA-3955, PTA-4113, or PTA-4257;
(b) a mutant, recombinant, or genetically engineered derivative of the wheat plant with ATCC Patent Deposit Designation Number PTA-3955, PTA-4113, or PTA-4257;
(c) any descendent of the plant with ATCC Patent Deposit Designation Number PTA-3955, PTA-4113, or PTA-4257; and
(d) a wheat plant that is the descendent of any one or more of these plants.

9. The method of claim 7, wherein said first parent wheat plant comprises the herbicide resistance characteristics of the wheat plant having ATCC Patent Deposit Designation Number PTA-3955, PTA-4113, or PTA-4257.

10. The method of claim 7, wherein said first parent wheat plant is the pollen donor, said second parent wheat plant is the pollen acceptor for said crossing, and said F1 progeny is produced on said second parent wheat plant.

11. The method of claim 7, wherein said second parent wheat plant is the pollen donor, said first parent wheat plant is the pollen acceptor for said crossing, and said F1 progeny is produced on said first parent wheat plant.

12. The method of claim 7, wherein said high protein wheat plant is selected by applying an effective amount of an AHAS-inhibiting herbicide to the F1 progeny so as to select for wheat plants with increased resistance to an AHAS-inhibiting herbicide.

13. The method of claim 7, wherein said first parent wheat plant is heterozygous or homozygous for said AHASL1A S653N gene.

14. The method of claim 7, wherein the F1 progeny produced by said crossing is grown and allowed to self-pollinate so as to produce F2 progeny.

15. The method of claim 14, wherein said high protein wheat plant is selected from said F2 progeny by applying an effective amount of an AHAS-inhibiting herbicide to the F2 progeny so as to select for at least one wheat plant with increased resistance to an AHAS-inhibiting herbicide.

16. The method of claim 1, wherein said wheat AHASL1A S653N gene is introduced into said high protein wheat plant by mutagenesis and selection for wheat plants comprising resistance to an effective amount of an AHAS-inhibiting herbicide.

17. The method of claim 16, further comprising selecting for wheat plants comprising the AHASL1A S653N gene.

18. The method of claim 1, wherein said wheat AHASL1A S653N gene is introduced into said high protein wheat plant by transformation comprising introducing into at least one cell of a wheat plant a polynucleotide construct comprising a wheat AHASL1A S653N polynucleotide operably linked to a promoter that drives expression in a plant cell so as to produce a transformed wheat cell and regenerating said transformed wheat cell into a transformed wheat plant, wherein the transformed wheat plant is said high protein wheat plant.

19. The method of claim 18, further comprising applying an effective amount of an AHAS-inhibiting herbicide to the transformed wheat cell so as to select for a transformed wheat cell comprising increased resistance to an AHAS-inhibiting herbicide.

20. The method of claim 18, wherein said promoter is selected from the group consisting of constitutive promoters and seed-preferred promoters.

21. The method of claim 1, wherein said high protein wheat plant has enhanced resistance to at least one AHAS-inhibiting herbicide selected from the group consisting of an imidazolinone herbicide, a sulfonylurea herbicide, a triazolopyrimidine herbicide, a pyrimidinyloxybenzoate herbicide, and a sulfonylamino-carbonyltriazolinone herbicide.

22. The method of claim 21, wherein said imidazolinone herbicide is selected from the group consisting of: [2-(4-isopropyl-4-methyl-5-oxo-2-]imidazolin-2-yl)-nicotinic acid, 2-(4-isopropyl)-4-methyl-5-oxo-2-imidazolin-2-yl)-3-quinolinecarboxylic acid, [5-ethyl-2-(4-isopropyl-4-methyl-]-5-oxo-2-imidazolin-2-yl)-nicotinic acid, 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, and a mixture of methyl 6-(4-isopropyl-4-methyl-5-oxo-2-imidazolin-2-yl)-m-toluate, methyl[2-(4-]isopropyl-4-methyl-5-oxo-2-imidazolin-2-yl)-p-toluate, and mixture thereof.

23. The method of claim 1, wherein the species of the high protein wheat plant is Triticum aestivum.

24. A method for making a wheat plant which produces high protein grain, said method comprising the steps of:
(a) introducing into a wheat plant at least one copy of a wheat AHASL1A S653N gene by cross pollination, wherein said cross pollination comprises crossing a first parent wheat plant to a second parent wheat plant so as to produce at least one F1 progeny plant, wherein said first parent wheat plant comprises at least one copy of said AHASL1A S653N gene and wherein said high protein wheat plant is descended from said first and said second parent wheat plants;
(b) growing the F1 progeny plant, or a descendent plant thereof comprising the AHASL1A S653N gene, to produce grain; and
(c) determining the protein content of grain produced by the F1 progeny plant or the descendent plant, wherein the F1 progeny plant or the descendent plant produces grain having an increased level of protein when compared to grain produced by a wheat plant lacking said wheat AHASL1A S653N gene.

25. The method of claim 24, wherein second parent wheat plant lacks at least one copy of said AHASL1A S653N gene.

26. A method for making a wheat plant which produces high protein grain, said method comprising the steps of:
(a) introducing into a wheat plant at least one copy of a wheat AHASL1A S653N gene by mutagenesis and selection for a wheat plant comprising resistance to an effective amount of an AHAS-inhibiting herbicide;
(b) growing the selected wheat plant, or a descendent plant thereof comprising the AHASL1A S653N gene, to produce grain; and
(c) determining the protein content of grain produced by the selected wheat plant or the descendent plant, wherein the selected wheat plant or the descendent plant produces grain having an increased level of protein when compared to grain produced by a wheat plant lacking said wheat AHASL1A S653N gene.

27. The method of claim 26, wherein said wheat plant lacks at least one copy of said AHASL1A S653N gene prior to step (a).

28. A method for making a wheat plant which produces high protein grain, said method comprising the steps of:
(a) transforming at least one cell of a wheat plant with a polynucleotide construct comprising a wheat AHASL1A S653N polynucleotide operably linked to a promoter that drives expression in a plant cell so as to produce a transformed wheat cell;
(b) regenerating said transformed wheat cell into a transformed wheat plant;
(c) growing the transformed wheat plant, or a descendent plant thereof comprising the AHASL1A S653N gene, to produce grain; and
(d) determining the protein content of grain produced by the transformed wheat plant or the descendent plant, wherein the transformed wheat plant or the descendent plant produces grain having an increased level of protein when compared to grain produced by a wheat plant lacking said wheat AHASL1A S653N gene.

29. The method of claim 28, wherein said wheat plant lacks at least one copy of said AHASL1A S653N gene prior to step (a).
